(19) United States
(12) Patent Application Publication
(10) Pub. No.: US 2025/0281383 A1
(43) Pub. Date: Sep. 11, 2025

(54) NON-THERAPEUTIC USE OF HYPERBRANCHED DEXTRINS AS A SOOTHING AGENT

(71) Applicant: ROQUETTE FRERES, Lestrem (FR)

(72) Inventors: Géraldine LOUVET-POMMIER, Lachelle (FR); Léon MENTINK, LILLE (FR); Daniel WILS, MORBECQUE (FR)

(21) Appl. No.: 18/852,852

(22) PCT Filed: Mar. 30, 2023
(86) PCT No.: PCT/EP2023/025144

§ 371 Date: May 21, 2025

(30) Foreign Application Priority Data
Mar. 30, 2022 (FR) FR2202904

(51) Int. Cl. A61K 8/73; A61Q 5/00; A61Q 11/00; A61Q 19/00

(52) U.S. Cl. CPC A61K 8/732; A61Q 5/00; A61Q 11/00; A61Q 19/00; A61Q 19/005

(57) ABSTRACT

The present invention relates to hyperbranched dextrins, preferentially hydrogenated hyperbranched dextrins, for use as a soothing agent as well as in a method for preventing or reducing or eliminating reactions of the skin and/or the scalp and/or the oral mucous membranes.

### TECHNICAL FIELD

The present invention relates to hyperbranched dextrins, preferentially hydrogenated hyperbranched dextrins, for use as a soothing agent and in a method for preventing or reducing or reversing cutaneous and oral mucosal reactions.

### PRIOR ART

The skin and oral mucosa constitute both a living anatomical barrier and a zone of exchange between the body and its environment, the effectiveness of which determines the maintenance of a good homeostatic balance. The skin comprises a superficial layer, the epidermis, which is made up of keratinized epithelial cells, and deeper layers, the dermis and hypodermis; each of these layers has specific properties that enable it to react and adapt to environmental conditions. Oral mucosa is structured in a similar way to the skin, with the difference that it does not have a surface layer of keratinized epithelial cells, but instead its surface is made up of non-keratinized epithelial cells.

Reactions of the skin (cutaneous) and oral mucosa can be triggered by the environment, e.g. cold, UV radiation, friction, food or drink, as well as by certain products and active ingredients present in cosmetic, dermocosmetic, dermatological, pharmaceutical, medicinal, veterinary or household compositions. These cutaneous reactions cause discomfort to the user's skin, scalp and oral mucosa, such as heating, tightness, tingling, itching and numbness. These cutaneous reactions are more pronounced on sensitive skin and scalps, and on sensitive mucous membranes. Cosmetic treatments can alleviate these feelings of discomfort associated with cutaneous and oral mucosal reactions by providing a soothing effect when the cosmetic product is applied.

There is therefore a need to identify soothing compounds that can also prevent, reduce and soothe cutaneous and oral mucosal reactions generated by the environment or cosmetic products.

Certain C-glycoside derivatives are known for their soothing properties and have been incorporated into cosmetic compositions. Those disclosed in document FR2902998 may be cited.

### SUMMARY

The applicant has demonstrated that a hyperbranched dextrin marketed under the reference Nutriose® HM 06 by Roquette soothes the sensations of heating and tightness caused by 20 hours of exposure of the skin of the forearms to sodium lauryl sulfate (SLS).

The present invention relates to the use of hyperbranched dextrins as a soothing agent.

The present invention also relates to the use of hyperbranched dextrins to prevent or reduce or eliminate cutaneous reactions and/or oral mucosal reactions.

The present invention also relates to a method of non-therapeutic care of the skin and/or scalp and/or oral mucosa comprising the application of hyperbranched dextrins to the skin and/or scalp and/or oral mucosa.

The present invention also relates to cosmetic compositions comprising hyperbranched dextrins in a cosmetically acceptable medium.

These compositions are suitable for all skin types and are particularly suitable for sensitive skin and/or scalps and sensitive oral mucosa.

### DETAILED DESCRIPTION

### Hyperbranched Dextrin

According to a first embodiment, the dextrins useful for the non-therapeutic use which is the object of the present application are hyperbranched dextrins.

By “dextrin”, the applicant means glucose polymers obtained from granular starch by pyroconversion, usually by the action of an acid in a generally dry environment, that is to say on granules in the form of dry solid particles containing residual moisture imposed by physical equilibrium conditions at a given temperature and pressure. Thus, maltodextrins and pyrogenic dextrins fall within the general family of dextrins useful for the invention when they have been modified to be branched.

In the context of the invention, by “dextrin”, the applicant also means glucose polymers derived from the acid or enzymatic liquefaction or hydrolysis of starch, and generally referred to as “maltodextrin”, “starch hydrolysate” or “glucose syrup”, when these polymers have been further chemically or enzymatically modified, in particular by branching enzymes, to have glucosidic bonds between the anhydroglucose molecules, significantly different in nature from those of the starch from which they are derived. Maltodextrins, starch hydrolysates, glucose syrups and pyrogenic dextrins thus fall into the general family of dextrins useful for the invention, as they differ in terms of carbohydrate bonds from the starch from which they are derived, by comprising “atypical” bonds.

By “hyperbranched dextrin”, the applicant means a “dextrin” having glucosidic bonds between the anhydroglucose molecules, significantly different in nature and quantity from those naturally constituting the starch from which it is derived. It comprises glucosidic bonds between anhydroglucose molecules that are naturally present, 1-6 bonds, but in greater quantity, and glucosidic bonds not naturally present in starch, known as “atypical”, 1-3 and 1-2 bonds. Thus, a hyperbranched dextrin is a dextrin with a high proportion of branching glucosidic bonds relative to the total glucosidic bonds present in said dextrin. The term “hyperbranched” can also be replaced by “highly branched”.

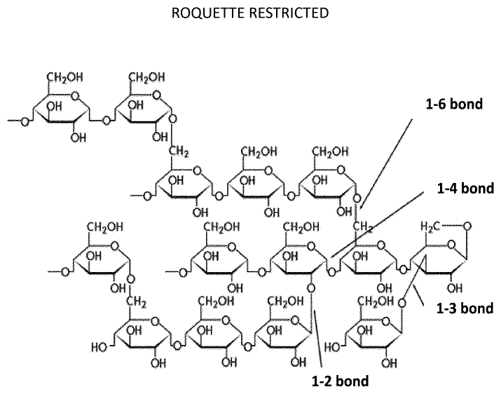

By “high proportion” we mean a proportion of branching glucosidic bonds greater than or equal to 5% of the sum of branching glucosidic bonds and linear glucosidic bonds, preferentially greater than or equal to 10%, more preferentially greater than or equal to 20%, more preferentially greater than or equal to 30%, more preferentially greater than or equal to 40%, more preferentially greater than or equal to 50%. Glucosidic branching bonds are bonds that generate non-linear chains, in higher proportions than normal for native starch. The glucosidic bonds that generate non-linear chains are glucosidic 1-6, 1-3, and 1-2 bonds, as shown in FIG. 1 [FIG. 1]. 1-3 and 1-2 bonds are atypical glucosidic bonds. Normal values for these bonds in native starch are around 5% for 1-6 bonds, and around 0% for 1-2 and 1-3 bonds. The remainder is made up of 1-4 glucosidic bonds, that is to say around 95%. The 1-4 bond is a bond that creates a linear bond.

For 1-6 glucosidic bonds, characteristic values of hyperbranched dextrins useful for the invention are values greater than 5% relative to the sum of 1-2, 1-3, 1-4 and 1-6 bonds, preferentially greater than or equal to 10%, with respect to the sum of 1-2, 1-3, 1-4 and 1-6 bonds, and more preferentially greater than or equal to 12% with respect to the sum of 1-2, 1-3, 1-4 and 1-6 bonds, and most preferentially greater than or equal to 15% with respect to the sum of 1-2, 1-3, 1-4 and 1-6 bonds.

Thus, according to one embodiment, hyperbranched dextrins comprise at least 5%, preferentially at least 10%, preferentially at least 12%, preferentially at least 15% of 1-6 glucosidic bonds relative to the sum of alpha 1-2, 1-3, 1-4 and 1-6 bonds.

For 1-2 and 1-3 glycosidic bonds, values greater than 1% based on the sum of 1-2, 1-3, 1-4 and 1-6 bonds, preferentially greater than or equal to 5% based on the sum of 1-2, 1-3, 1-4 and 1-6 bonds, and more preferentially greater than or equal to 10% based on the sum of 1-2, 1-3, 1-4 and 1-6 bonds, are useful for the invention.

Thus, according to one embodiment, hyperbranched dextrins comprise at least 1%, preferentially at least 5%, preferentially at least 10%, and preferentially at least 20% of 1-2 glucosidic bonds relative to the sum of 1-2, 1-3, 1-4 and 1-6 bonds.

According to one embodiment, hyperbranched dextrins comprise at least 1%, preferentially at least 5%, preferentially at least 10%, and preferentially at least 20% of 1-3 glucosidic bonds relative to the sum of 1-2, 1-3, 1-4 and 1-6 bonds.

For 1-4 glycosidic bonds, values less than 70%, preferentially less than 60%, preferentially less than 50% based on the sum of 1-2, 1-3, 1-4 and 1-6 bonds and preferentially between 42 and 50% based on the sum of 1-2, 1-3, 1-4 and 1-6 bonds, are useful for the invention.

Thus, according to one embodiment, hyperbranched dextrins comprise at most 70%, preferentially at most 50% of 1-4 glucosidic bonds relative to the sum of 1-2, 1-3, 1-4 and 1-6 bonds.

Preferentially, hyperbranched, preferably hyperbranched and hydrogenated dextrins useful for the invention have a 1-6 glucosidic bond content of between 5% and 40%, preferentially between 10% and 30%, more preferentially between 12% and 22%, and most preferentially between 15 and 20%, based on the sum of 1-2, 1-3, 1-4 and 1-6 bonds.

According to one embodiment, the hyperbranched dextrins, preferably hyperbranched and hydrogenated dextrins, useful to the invention have:
from 5% to 40%, preferentially between 10% and 30%, more preferentially between 12% and 22%, and most preferentially between 15 and 20% of 1-6 glucosidic bonds relative to the sum of 1-2, 1-3, 1-4 and 1-6 bonds,
from 42 to 50% of 1-4 glucosidic bonds relative to the sum of 1-2, 1-3, 1-4 and 1-6 bonds,
from 1 to 20% of 1-3 glucosidic bonds relative to the sum of 1-2, 1-3, 1-4 and 1-6 bonds,
from 1 to 20% of 1-2 glycosidic bonds relative to the sum of 1-2, 1-3, 1-4 and 1-6 bonds.

According to one embodiment, the hyperbranched dextrins, preferably hyperbranched and hydrogenated dextrins, useful to the invention have:
at least 5% of 1-6 glucosidic bonds relative to the sum of 1-2, 1-3, 1-4 and 1-6 bonds, preferentially at least 10%, more preferentially at least 12%, and most preferentially at least 15%,
no more than 70% of 1-4 glycosidic bonds, relative to the sum of 1-2, 1-3, 1-4 and 1-6 bonds, preferentially no more than 60%, and most preferentially no more than 50%,
at least 1% of 1-3 glucosidic bonds relative to the sum of 1-2, 1-3, 1-4 and 1-6 bonds, preferentially at least 5%, more preferentially at least 10%, and most preferentially at least 20%,
at least 1% of 1-2 glucosidic bonds relative to the sum of 1-2, 1-3, 1-4 and 1-6 bonds, preferentially at least 5%, more preferentially at least 10%, and most preferentially at least 20%.

According to one embodiment, the hyperbranched dextrins, preferably hyperbranched and hydrogenated dextrins, useful to the invention have:
from 5% to 40%, preferentially from 10% to 30%, more preferentially from 12% to 22%, and most preferentially from 15 to 20% of 1-6 glucosidic bonds relative to the sum of 1-2, 1-3, 1-4 and 1-6 bonds,
from 42 to 70%, preferentially from 42 to 60% of 1-4 glucosidic bonds relative to the sum of 1-2, 1-3, 1-4 and 1-6 bonds,
from 1 to 20%, preferentially from 1 to 10%, more preferentially from 5 to 15%, and most preferentially from 5 to 10%, of 1-3 glucosidic bonds relative to the sum of 1-2, 1-3, 1-4 and 1-6 bonds, —from 1 to 20%, preferentially from 1 to 10%, more preferentially from 5 to 15%, and most preferentially from 5 to 10%, of 1-2 glucosidic bonds relative to the sum of 1-2, 1-3, 1-4 and 1-6 bonds.

Apart from the use of a well-known pyroconversion process applied to granular starch to prepare dextrins, that is to say by using an acid on granular starch, typically at high temperature in a dry environment, these atypical contents of branching bonds in a starch can also be obtained by the action of a so-called “branching” or “re-branching” enzyme on a liquefied starch, such as a maltodextrin, a starch hydrolysate or a glucose syrup, optionally previously or subsequently hydrogenated.

These atypical bond contents can therefore be obtained both by the action of a “branching” or “re-branching” enzyme on a liquefied starch and/or by the action of an acid, typically at high temperature in a dry medium, on a granular or liquefied starch.

The content of 1-2, 1-3, 1-4 and 1-6 glucosidic bonds can be determined using the conventional methylation technique described in HAKOMORI, S., 1964, J. Biochem., 55, 205. “A rapid Permethylation of Glycolipid, and Polysaccharide Catalyzed by Methylsulfinyl Carbanion in Dimethyl Sulfoxide”. This method makes it possible to chemically characterize glucosidic bonds by differentiating between free OH groups and bonded groups. This is a destructive method comprising the steps of methylation, hydrolysis, reduction with NaBD4, acetylation and analysis by mass spectrometry.

According to one embodiment, said hyperbranched dextrins have a number-average molecular weight Mn of less than or equal to 10,000 g/mol, preferentially less than or equal to 4,500 g/mol, more preferentially between 1,000 and 3,500 g/mol, even more preferentially between 1,500 and 3,500 g/mol, and most preferentially between 1,800 and 3,200 g/mol.

According to one embodiment, said hyperbranched dextrins have a polymolecularity index, the ratio of the weight-average molar mass to the number-average molar mass, noted IP, of less than or equal to 15, preferentially less than or equal to 10, more preferentially less than or equal to 5, and most preferentially less than or equal to 3.

Mn, Mw and IP values are measured by steric exclusion chromatography, based on the size-selective retention of solute molecules due to their penetration or non-penetration into the pores of the stationary phase. The size exclusion chromatography columns used are PSS SUPREMA 100 and PSS SUPREMA 1000, connected in series and coupled to a light scattering detector.

According to one embodiment, said hyperbranched dextrins have a reducing sugar content of less than or equal to 20%, preferentially less than or equal to 15%, more preferentially less than or equal to 10%, even more preferentially less than or equal to 6%, and most preferentially less than or equal to 3% relative to the total mass of the hyperbranched dextrin. The reducing sugar content, expressed as glucose by weight in relation to the dry weight of the product analyzed, is determined by the BERTRAND method.

According to one embodiment, hyperbranched dextrins useful for the invention have a relatively high content of residual 1-4 glucosidic bonds. This content of 1-4 glucosidic bonds is between 42 and 50% relative to the sum of 1-2, 1-3, 1-4 and 1-6 bonds, in combination with a content of 1-6 glucosidic bonds between 12 and 22% based on the sum of 1-2, 1-3, 1-4 and 1-6 bonds. Preferentially, hyperbranched dextrins useful for the invention have a 1-4/1-6 glucosidic bond ratio of between 1.9 and 4.2, and in particular between 2.3 and 3.5.

According to one embodiment, hyperbranched dextrins useful for the invention have a content of 1-3 glucosidic bonds of between 1 and 10% relative to the sum of 1-2, 1-3, 1-4 and 1-6 bonds, in combination with a content of 1-4 glucosidic bonds of between 42 and 50% relative to the sum of 1-2, 1-3, 1-4 and 1-6 bonds. Preferentially, hyperbranched dextrins useful for the invention have a 1-4/1-3 glucosidic bond ratio of between 0.02 and 0.24, and in particular between 0.1 and 0.2. According to one embodiment, hyperbranched dextrins useful for the invention have a content of 1-2 glucosidic bonds of between 1 and 10% in combination with a content of 1-4 glucosidic bonds of between 42 and 50%. Preferentially, hyperbranched dextrins useful for the invention have a 1-4/1-2 glucosidic bond ratio of between 0.02 and 0.24, and in particular between 0.1 and 0.2.

According to one embodiment, the hyperbranched dextrins useful to the invention have:
from 5% to 40% of 1-6 glucosidic bonds in relation to the sum of 1-2, 1-3, 1-4 and 1-6 bonds,
a reducing sugar content less than or equal to 20% relative to the total mass of hyperbranched dextrin, —a polymolecularity index less than or equal to 15,
and a number-average molecular weight Mn less than or equal to 4500 g/mol.

According to one embodiment, the hyperbranched dextrins useful to the invention have:
from 10% to 30% of 1-6 glucosidic bonds in relation to the sum of 1-2, 1-3, 1-4 and 1-6 bonds,
from 42 to 70% of 1-4 glucosidic bonds relative to the sum of 1-2, 1-3, 1-4 and 1-6 bonds,
a reducing sugar content less than or equal to 10% relative to the total mass of hyperbranched dextrin,
a polymolecularity index less than or equal to 10,
and a number-average molecular weight Mn between 1000 and 3500 g/mol.

According to one embodiment, the hyperbranched dextrins useful to the invention have:
from 12% to 22% of 1-6 glucosidic bonds in relation to the sum of 1-2, 1-3, 1-4 and 1-6 bonds,
from 42 to 50% of 1-4 glucosidic bonds relative to the sum of 1-2, 1-3, 1-4 and 1-6 bonds,
from 1 to 20% of 1-3 glycosidic bonds relative to the sum of 1-2, 1-3, 1-4 and 1-6 bonds,
from 1 to 20% of 1-2 glycosidic bonds relative to the sum of 1-2, 1-3, 1-4 and 1-6 bonds,
a reducing sugar content less than or equal to 6% relative to the total mass of hyperbranched dextrin,
a polymolecularity index less than or equal to 5,
and a number-average molecular weight Mn between 1500 and 3000 g/mol.

Hyperbranched dextrins useful for the invention are commercially available, such as Roquette®'s “Nutriose® FM 06”, “Nutriose® FM 10” and “Nutriose® FM15S” products, or Tate & Lyle's “Promitor® Soluble fiber” product line.

Other commercial products available under the names “STA-LITE® Polydextrose” from Tate & Lyle, or “Oliggo-fiber®” from Cargill, are hyperbranched polysaccharides or oligosaccharides which, as they stand or after possible hydrogenation, could conceivably have a soothing property like the hyperbranched dextrins disclosed in the present application.

### Hyperbranched and Hydrogenated Dextrins

According to a second embodiment, the dextrins useful for the non-therapeutic use which is the object of the present application are hyperbranched and hydrogenated dextrins. Such dextrins can be obtained by subjecting hyperbranched dextrins according to the object of the present application to hydrogenation, or by applying a “branching” or “re-branching” method to a previously hydrogenated dextrin. Hydrogenation can, for example, be achieved by subjecting an aqueous solution of hyperbranched dextrin to hydrogen gas in the presence of a catalyst such as Raney nickel.

An additional feature can be added to the above-mentioned embodiments for hyperbranched dextrins, namely a reducing sugar content of less than or equal to 5% by weight, preferentially less than or equal to 3% by weight, more preferentially less than or equal to 2% by weight, more preferentially less than or equal to 1% by weight, more preferentially less than or equal to 0.5% by weight, and most preferentially less than or equal to 0.15% by weight.

According to one embodiment, the hyperbranched and hydrogenated dextrins useful to the invention have:
from 5% to 40% of 1-6 glucosidic bonds in relation to the sum of 1-2, 1-3, 1-4 and 1-6 bonds,
a reducing sugar content less than or equal to 4% relative to the total mass of hyperbranched dextrin,
a polymolecularity index less than or equal to 15,
and a number-average molecular weight Mn less than or equal to 4500 g/mol.

According to one embodiment, the hyperbranched and hydrogenated dextrins useful to the invention have:
from 10% to 30% of 1-6 glucosidic bonds in relation to the sum of 1-2, 1-3, 1-4 and 1-6 bonds,
from 42 to 70% of 1-4 glucosidic bonds relative to the sum of 1-2, 1-3, 1-4 and 1-6 bonds,
a reducing sugar content less than or equal to 3% relative to the total mass of hyperbranched dextrin,
a polymolecularity index less than or equal to 10,
and a number-average molecular weight Mn between 1000 and 3500 g/mol.

According to one embodiment, the hyperbranched and hydrogenated dextrins useful for the invention have: —from 12% to 22% of 1-6 glucosidic bonds relative to the sum of 1-2, 1-3, 1-4 and 1-6 bonds,
from 42 to 50% of 1-4 glucosidic bonds relative to the sum of 1-2, 1-3, 1-4 and 1-6 bonds,
from 1 to 20% of 1-3 glycosidic bonds relative to the sum of 1-2, 1-3, 1-4 and 1-6 bonds,
from 1 to 20% of 1-2 glycosidic bonds relative to the sum of 1-2, 1-3, 1-4 and 1-6 bonds,
a reducing sugar content less than or equal to 2% relative to the total mass of hyperbranched dextrin,
a polymolecularity index less than or equal to 5,
and a number-average molecular weight Mn between 1500 and 3000 g/mol.

Hyperbranched and hydrogenated dextrins useful for the invention are commercially available, such as Roquette's “Nutriose® HM 06”.

### Soothing Agent

The object of the present invention is the non-therapeutic use of hyperbranched dextrins, preferentially hyperbranched and hydrogenated dextrins, as a soothing agent.

A soothing agent is an agent that prevents the onset of cutaneous reactions and/or oral mucosal reactions, or reduces the intensity of these reactions or eliminates them.

Thus, according to one embodiment, the object of the invention is the non-therapeutic use of hyperbranched dextrins, preferentially hyperbranched and hydrogenated dextrins, as a soothing agent for cutaneous reactions and/or oral mucosal reactions.

“Cutaneous” refers to the skin, that is to say the outer covering of the human body and the body of vertebrate animals, including the human scalp. Cutaneous reactions cause discomfort on the user's skin and scalp. Cutaneous reactions include warming, tightness, tingling, numbness and itching. These reactions are non-pathological cutaneous reactions.

Thus, according to one embodiment, the object of the invention is the non-therapeutic use of hyperbranched dextrins, preferentially hyperbranched and hydrogenated dextrins, as a soothing agent for cutaneous reactions of the skin and/or scalp.

According to one embodiment, cutaneous reactions are selected from warming, tightness, tingling, numbness and itching.

“Oral mucosa” refers to the lips, tongue, floor of the tongue, gums, inner surface of the cheeks and lips, roof of the mouth and soft palate in humans and vertebrate animals.

Reactions of the oral mucosa cause discomfort in the lips, gums, inner cheeks or roof of the mouth. Reactions of the oral mucosa include warming, burning, warming, tightness, tingling, numbness and itching. According to one embodiment, oral mucosal reactions are selected from burning, tingling or numbness.

These cutaneous reactions are increased in individuals with sensitive skin or scalps. However, unlike allergic skin, this reactivity is not the result of an immunological process. Sensitive skin is defined by a particular reactivity. This cutaneous reactivity conventionally translates into the manifestation of previously defined signs of discomfort such as heating, tightness, tingling, numbness and itching. These cutaneous reactions may be in response to contact with a triggering element. These cutaneous reactions can occur on all skin types, but are more frequent on skin in a state of hypersensitivity, designated by the term “sensitive skin”, which corresponds to a state of skin with increased sensitivity to external stimuli, that is to say greater sensitivity than the population average, or greater than usual for an individual.

“Sensitive scalp” refers to scalps for which itching and/or tingling and/or heating sensations are essentially triggered by local factors such as rubbing, soap, surfactants, hard water with a high lime content, shampoos or lotions. These sensations are also sometimes triggered by factors such as the environment, emotions and/or food. Scalp erythema, hyperseborrhea and dandruff are frequently associated with the above signs.

According to one embodiment, cutaneous reactions are cutaneous reactions of sensitive skin and scalps.

The term “sensitive oral mucosa” refers to oral mucosa showing an exacerbated sensitivity compared to the average sensitivity of a population, or compared to the usual sensitivity of an individual. This sensitivity may be revealed by contact with surfactants such as sodium lauryl sulfate, alcohols such as ethanol, whitening agents such as peroxides, or active ingredients or essential oils used in mouthwash, gel or toothpaste compositions.

According to one embodiment, cutaneous reactions and oral mucosal reactions are induced by endogenous or exogenous stress.

These cutaneous reactions can be induced by exogenous stresses such as
chemicals or formulation ingredients,
mechanical aggressions chosen from rubbing, shaving, cuts, micro-cuts, depilation, abrasion, scrubbing, frequent washing, hard water with a high lime concentration,
environmental factors such as pollution, UV rays and exposure to the sun,
thermal aggression, chosen from overexposure to the sun and exposure to a source of heat or cold
foods and beverages that are abrasive or chemically aggressive to the oral mucosa, etc.

Endogenous factors such as hormonal secretions, age, stress, perspiration, etc. can also induce a cutaneous or oral mucosal reaction.

Prevention, Reduction or Elimination of Cutaneous and/or Oral Mucosal Reactions

The present invention also relates to the non-therapeutic use of hyperbranched dextrins, preferentially hyperbranched and hydrogenated dextrins, to prevent or reduce or eliminate cutaneous reactions and/or oral mucosal reactions.

According to one embodiment, cutaneous reactions are selected from warming, tightness, tingling, numbness and itching.

According to one embodiment, oral mucosal reactions are selected from burning, warming, tightness, tingling, numbness and itching.

Prevention of cutaneous or oral mucosal reactions means preventing the onset of one or more cutaneous or oral mucosal reactions, delaying the onset of one or more cutaneous or oral mucosal reactions, and preventing the worsening of one or more cutaneous or oral mucosal reactions.

Reduction in cutaneous or oral mucosal reactions means a reduction in one or more cutaneous or oral mucosal reactions. For example, reducing cutaneous reactions means reducing the intensity of warming, tightness, tingling, numbness and itching. For example, a reduction in oral mucosal reactions means a reduction in the intensity of warming, burning, tightness, tingling, numbness, itching and, particularly when brushing teeth, a reduction in the intensity of dry mouth sensations, nausea and retching.

The disappearance of cutaneous or oral mucosal reactions means the absence of one or more cutaneous or oral mucosal reactions.

According to one embodiment, the skin and scalp are sensitive skin and scalp.

According to one embodiment, cutaneous reactions and oral mucosal reactions are induced by endogenous or exogenous stress as previously defined.

### Cosmetic Method

The present invention also relates to a method of non-therapeutic care of the skin and/or scalp and/or oral mucosa comprising the application of hyperbranched dextrins, preferentially of hyperbranched and hydrogenated dextrins to the skin and/or scalp and/or oral mucosa.

Hyperbranched dextrins can be applied in the form of a cosmetic composition comprising them.

According to one embodiment, the hyperbranched dextrins or the cosmetic composition comprising them is/are to be applied to the areas to be soothed of an individual exhibiting one or more cutaneous or oral mucosal reactions and is/are optionally left in contact for several minutes or several hours and is/are optionally rinsed off. As a result, this method can be carried out during or after irritation, and can be repeated or renewed as often as necessary, over periods ranging from a few days to several months or even years.

Thus, according to one embodiment, the method comprises applying hyperbranched dextrins or the cosmetic composition comprising them to the skin and/or scalp and/or oral mucosa exhibiting one or more cutaneous reactions or oral mucosal reactions.

According to one embodiment, the method comprises an additional step wherein the compound is left in contact with the skin and/or scalp and/or oral mucosa.

According to one embodiment, the method comprises an additional rinsing step.

Alternatively, the hydrogenated hyperbranched dextrins or the cosmetic composition comprising them may be applied prior to the application of a cosmetic or dermatological treatment.

According to one embodiment of the invention, such a prevention method is used in cosmetic or dermatological pre-treatment, in order to prevent the onset of one or more cutaneous and/or oral mucosal reaction(s) likely to occur during exposure to cold, heat, sunlight, a source of pollution, foodstuffs, etc., or during the application of a subsequent cosmetic or dermatological treatment such as the application of a cosmetic or dermatological composition comprising one or more irritant agent(s), coloring, bleaching, perming, straightening, peeling, anti-rosacea treatment, etc.

Thus, the invention also relates to a non-therapeutic skin and/or scalp and/or oral mucosa care method comprising at least two steps: a first pre-treatment step, intended to prevent the occurrence of one or more cutaneous reactions and/or oral mucosal reactions, and which comprises the application to the skin and/or scalp and/or oral mucosa of hyperbranched dextrins or the cosmetic composition comprising them, and a second step, subsequent to said first step, of applying a cosmetic or dermatological treatment.

According to one embodiment of the invention, the hyperbranched dextrins or the cosmetic composition comprising them can be applied in anticipation of endogenous and/or exogenous stress.

The endogenous and/or exogenous stresses are as previously defined.

### Cosmetic Composition

Hyperbranched dextrins, preferentially hydrogenated hyperbranched dextrins, can be incorporated into a cosmetic composition.

Thus, the present invention also relates to a cosmetic composition comprising hyperbranched dextrins and a cosmetically acceptable medium.

“Cosmetically acceptable” is understood to mean a medium which does not present deleterious side effects and in particular under normal usage conditions does not produce redness, inflammation, heating, tightness or tingling unacceptable for a user of cosmetic products. The medium is thus compatible with the skin, scalp and oral mucosa of humans and animals.

According to one embodiment, the compound according to the invention can be incorporated into a cosmetic composition containing one or more compounds liable to cause skin irritation.

According to one embodiment, the hyperbranched dextrins or the cosmetic composition comprising them is/are applied to non-pathological skin exhibiting one or more non-pathological cutaneous reactions.

According to one embodiment, the content of hyperbranched dextrins is between 0.1 and 50%, preferentially between 0.5 and 20%, more preferentially 1 and 10% by weight, and most preferentially between 1.5 and 7.5%, and generally 2 to 5% relative to the total weight of the composition.

### Forms of Administration

The cosmetic composition according to the invention may be in any pharmaceutical form normally used for topical application such as aqueous, hydroalcoholic or oily solutions, lotion- or serum-type dispersions or solutions, milk-type emulsions of liquid or semi-liquid consistency, obtained by dispersion of a fatty phase in an aqueous phase (O/W) or vice-versa (W/O), or soft, semi-solid or solid suspensions or emulsions, like cream-type emulsions, aqueous or anhydrous gels, anhydrous compositions, solid compositions, microemulsions, microcapsules, microparticles, or ionic and/or nonionic vesicular dispersions. For the purposes of the present invention, “topical application” means application to a body surface, such as the skin or mucous membranes.

These compositions are prepared according to the usual methods known to the person skilled in the art.

This composition, when applied to the skin, may be more or less fluid and have the appearance of a cream, an emulsion or microemulsion, a salve, a milk, a lotion, a serum, a paste, a foam, a mask, a fluid, a balm, an oil, a gel, an ointment, a mouthwash, a toothpaste, a gum paste. It can optionally be applied to the skin in aerosol form. It can also take a solid form, for example in stick or patch form, or in the form of dry soaps or cleansing bars.

It can be used as a skincare product, particularly for leave-on application, such as a day or night cream for facial and/or body skin, as a cleanser, as a make-up product, as a deodorant, as a shaving product, as an aftershave product, as an after-sun product, as a sun protection product, as a lip balm to protect lips from the cold and/or the sun and/or the wind, etc. It can also be used as a rinse-off skincare product.

The composition according to the invention can also be a scalp and hair care composition, particularly in pharmaceutical forms for rinse-off application, such as shampoos, hair conditioners, hair masks, serums, foams, balms, creams, sprays, conditioners, detanglers, hair creams, mousses, balms, creams, sprays, conditioners, detanglers, hair creams, and also in the form of treatment lotions, dye compositions, coloring shampoos, perm compositions, anti-hair loss lotions or gels, anti-parasite shampoos, conditioners, etc. The composition according to the invention can also be a scalp and hair care composition, particularly in leave-on pharmaceutical form.

### Formulation Ingredients

The cosmetic composition according to the invention may also comprise a solvent chosen based on the various ingredients and the form of administration.

According to one embodiment, the composition according to the invention may comprise an aqueous phase comprising water and optionally one or more water-miscible organic solvents.

The term “water-soluble solvent” refers to a compound that is liquid at room temperature and miscible with water (miscibility in water greater than 50% by weight at 25° C. and atmospheric pressure).

Water-soluble solvents that can be used in the compositions according to the invention can be volatile.

Among the water-soluble solvents that can be used in the compositions in accordance with the invention, mention may be made of monoalcohols having from 1 to 5 carbon atoms such as ethanol and isopropanol, C₃-C₄ ketones and C₂-C₄ aldehydes and polyols, in particular such as glycerol or glycerin, sorbitol or isosorbide.

The cosmetic composition may also comprise, aside from the hyperbranched dextrins:

### One or More Humectants

The humectant(s) will be chosen from polyols and/or esters of fatty acids and of polyethylene glycol.

“Polyols” is understood to mean any molecule having in its structure at least two free hydroxy (—OH) groups. These polyols are preferably liquid at room temperature (25° C.).

Typically, the polyol will be selected from maltitol, mannitol, xylitol, erythritol, sorbitol, isosorbide, glycerol or glycerin, glucose, sucrose, polydextrose, hydrogenated glucose syrups, dextrins, maltodextrins, glucose syrups, and mixtures thereof.

Examples include Beauté by Roquette® PO 071 (INCI: Sorbitol), Beauté by Roquette® PO 260 (INCI: Mannitol), Beauté by Roquette® PO 370 (INCI: Xylitol), Beauté by Roquette® PO 455 (INCI: Hydrogenated starch hydrolysate), Beauté by Roquette® PO 500 (INCI: Isosorbide), all sold by ROQUETTE, glycerin (INCI: glycerin) sold by COOPER, propylene glycol (INCI: propylene Glycol) sold by COOPER, butylene Glycol (INCI: 1,3-BUTANEDIOL).

Among the esters of fatty acids and of polyethylene glycol, one may mention the product sold under the name Glucamate SSE-20 (INCI: PEG-20 METHYL GLUCOSE SESQUISTEARATE) by the company LUBRIZOL ADVANCED MATERIALS, Inc.

The composition comprises from 0.5% to 25% by weight of one or more humectant(s), preferably from 1% to 15% by weight, and even more preferably from 2% to 10% by weight of polyols, relative to the total weight of the composition.

### One or More Oils

For the purposes of the present invention, “oil” is understood to mean a compound which is liquid at room temperature (25° C.) and which, when it is introduced at an amount of at least 1% by weight into the water at 25° C., is not at all soluble in the water, or soluble to an extent of less than 10% by weight, relative to the weight of oil introduced into the water. According to one embodiment, the oil will be chosen from volatile oils, non-volatile oils and mixtures thereof. Preferably, these oils are vegetable or of vegetable origin.

The term “non-volatile oil” means an oil remaining on the skin, the scalp or the oral mucosa at room temperature and atmospheric pressure for at least several hours and especially having a vapor pressure of less than 10⁻³ mmHg (0.13 Pa).

Among non-volatile oils, mention may be made of fatty esters such as cetearyl isononoate, isotridecyl isononoate, isostearyl isostearate, isopropyl isostearate, isopropyl myristate, isopropyl palmitate, butyl stearate, hexyl laurate, isononyl isononate, 2-ethylhexyl palmitate, 2-hexyldecyl laurate, 2-octyldecyl palmitate, 2-octyldodecyl myristate or lactate, 2-diethylhexyl succinate, diisostearyl malate, triacetin, tricaprin, caprylic/capric acid triglycerides, coco caprate and caprylate mixture, benzoates of C12 to C15 alcohols, glycol esters such as butylene glycol cocoate, glyceryl triisostearate, tocopherol acetate, higher fatty acids such as myristic acid, palmitic acid, stearic acid, behenic acid, oleic acid, linoleic acid, linolenic acid or isostearic acid, higher fatty alcohols such as oleic alcohol, vegetable oils such as avocado oil, camellia oil, hazelnut oil, tsubaki oil, cashew nut oil, argan oil, soybean oil, grape seed oil, sesame oil, corn oil, wheatgerm oil, rapeseed oil, sunflower oil, cottonseed oil, jojoba oil, peanut oil, macadamia oil, sweet almond oil, olive oil and mixtures thereof.

These non-volatile oils may also be hydrocarbon or silicone type oils, such as paraffin oils, squalane, petroleum jelly, dimethyl siloxanes and mixtures thereof.

According to one embodiment, the non-volatile oil is chosen from stearic acid, jojoba oil (INCI: Simmondsia chinensis Seed Oil), grape seed oil (INCI: Vitis vinifera seed oil), macadamia oil (INCI: Macadamia ternifolia seed oil), refined oleic sunflower oil (Helianthus annuus seed oil), the mixture of caprate and coco caprylate such as the product Miglyol Coco 810 (INCI: Coco-Caprylate/Caprate), sweet almond oil (INCI: Prunus Amygdalus Dulcis Oil), sesame oil (INCI: Sesamum indicum seed oil).

Volatile oil is intended to mean an oil which is capable of evaporating from the skin in less than one hour at room temperature and atmospheric pressure. The volatile oils may for example be selected from silicone oils or short-chain fatty acid triglycerides in order to reduce the greasy feel.

According to one embodiment, the oil(s) are present at a content ranging from 0.5% to 70% by weight, preferably from 1% to 60% by weight, preferably from 2% to 50% by weight, preferably 5% to 40%, preferably from to 30% by weight, relative to the total weight of the composition.

One or More Waxes and/or One or More Pasty Compounds.

“Wax” is intended to mean a fatty substance having reversible liquid-solid state change and having a melting point of greater than 25° C., generally between 30° C. and 90° C., and which may be rendered liquid under the preparation conditions of the composition and which has anisotropic crystalline organization in the solid state. The waxes used according to the invention may consist of polar or apolar waxes or a mixture of these two. “Apolar” is intended to mean a wax containing only carbon, hydrogen and/or phosphorus atoms, and in particular a hydrocarbon.

The polar waxes may be selected from animal waxes, vegetable waxes, and synthetic or silicone waxes containing polar groups such as esters. Mention may thus be made of carnauba wax, candelilla wax, beeswax (Cera alba), Chinese insect wax (Ericerus pela), Japan wax, sumac wax, montan wax, triesters of C8-C20 acids and glycerin, such as glyceryl tribehenate, acetylated glycol stearate, sold particularly by VEVY under the trade name CETACENE, and mixtures thereof. These waxes may in particular be used in predispersed form in an oil, as is the case of the mixture of candelilla wax and jojoba seed oil.

According to one embodiment, the wax(es) are present at a content ranging from 0.5 to 50% by weight, preferably from 1 to 25% by weight, preferably from 5 to 10% by weight, relative to the total weight of the composition. Preferably, these waxes are vegetable or of vegetable origin.

“Pasty compound” is understood to mean lipophilic fatty substances which, like waxes, are capable of undergoing a reversible liquid-solid state change and which have, in the solid state, anisotropic crystalline organization, but which differ from waxes in that they contain, at a temperature of 23° C., a liquid fraction and a solid fraction.

According to one embodiment, the pasty compound(s) are present at a content ranging from 0.5 to 50% by weight, preferably from 1 to 25% by weight, preferably from 5 to 10% by weight, relative to the total weight of the composition. These pasty compounds are preferably vegetable butters or butters of vegetable origin, such as shea butter or camellia butter.

According to one embodiment, the pasty compound(s) are present at a content ranging from 0.5 to 50% by weight, preferably from 1 to 25% by weight, preferably from 5 to 10% by weight, relative to the total weight of the composition.

### One or More Gelling Agent(s)

A gelling agent refers to a compound which, in the presence of a solvent, creates more or less strong intermacromolecular bonds, thus inducing a three-dimensional network which fixes said solvent. Gelling agents also refer to thickening or rheological agents that act on the viscosity and flow properties of an aqueous or fatty phase.

The gelling agent in particular makes it possible to increase the viscosity of the continuous phase, that is to say to adjust the viscosity of the continuous phase to a value ranging from 100 mPa·s to 20,000 mPa·s.

The gelling agent can be chosen from polymers of synthetic origin or of plant origin, preferentially of plant origin, chemically modified or not. It can thus be chosen from gums derived from plants such as gum arabic, konjac gum, guar gum or derivatives thereof; gums extracted from algae such as alginates; gums derived from microbial fermentation such as xanthans, for example, the product Keltrol CG (INCI: xanthan gum), sold by the company CP KELCO or the product Xanthan Gum FNCS-PC (INCI: xanthan gum) sold by the company JUNGBUNZLAUER INTERNATIONAL AG, mannans, scleroglucans or derivatives thereof; cellulose and derivatives thereof such as carboxymethylcellulose or hydroxyethylcellulose; starch and derivatives thereof such as native starches and modified starches, notably pregelatinized, acetylated, hydroxypropylated, carboxymethylated, cationic, octenylsuccinates, cross-linked starches, optionally modified several times; synthetic polymers such as polyacrylic acids or carbomers, and mixtures thereof.

Among the starches and starch-based blends, mention will be made of Beauté by Roquette® ST 118 (INCI: Carboxymethyl starch), Beauté by Roquette® ST 720 (INCI: Hydroxypropyl starch) and Beauté by Roquette® DS112 (INCI: Starchacetate (and) Hydroxyethylcellulose (and) Xanthan gum), all sold by ROQUETTE.

Among the carbomers, mention will for example be made of the product Carbopol Ultrez 30 (INCI: carbomer) from the company LUBRIZOL ADVANCED MATERIALS, Inc. or CARBOPOL ETD 2050 POLYMER (INCI: carbomer).

Mention will also be made of mixtures of gum arabic and xanthan gum such as the product Solagum AX (INCI: Acacia Senegal gum (and) Xanthan gum), sold by the company SEPPIC, mixtures of starch and cellulose derivatives such as the product Beauté by ROQUETTE® DS112 (INCI: Starch acetate (and) Hydroxyethylcellulose (and) Xanthan gum), sold by the company ROQUETTE.

The gelling agent may be a mineral gelling agent selected from magnesium and/or aluminum silicates. An example of such a mineral gelling agent is Veegum® Pure from Vanderbilt Minerals LLC, which is a magnesium aluminum silicate.

According to one embodiment, the gelling agent(s) are present at a content ranging from 0.1 to 50% by weight, preferably from 0.5 to 25% by weight, preferably from 0.3 to 15% by weight, relative to the total weight of the composition.

### One or More Binders

The term “binder” means compounds conferring increased cohesion of the cosmetic composition. This cohesion can be adjusted as a function of the amount and the chemical affinity of the binder relative to the ingredients of the cosmetic composition.

Typically, the binder will be chosen from caprylic/capric acid triglicerides such as the product Labrafac CC (INCI: capric/caprylic triglicerides) or Cetyl Dimethicone (INCI).

The binder(s) are be present in the composition according to the invention at a content ranging from 0.5% to 20%, preferably from 1 to 15%, preferably from 5 to 12%, by weight relative to the total weight of the composition.

### One or More Emulsifying Agent(s)

The cosmetic composition according to the invention may also comprise one or more oil-in-water (O/W) or water-in-oil (W/O) emulsifiers.

The oil-in-water (O/W) emulsifier is an emulsifying agent of HLB greater than or equal to 8 and selected from sorbitan esters which could be polyethoxylated, glycerol fatty acid esters, sucrose fatty acid esters or polyesters, polyethylene glycol fatty acid esters, polyether-modified polysiloxanes, polyethyleneglycol fatty alcohol ethers, alkyl polyglycosides and hydrogenated lecithin, fatty alcohols, sorbitan esters, this list being non-limiting, and mixtures thereof.

Examples include Montanov 68 (INCI: CETEARYL ALCOHOL (AND) CETEARYL GLUCOSIDE), sold by SEPPIC, Montanov L (INCI: C14-C22 Alcohols & C12-20 Alkyl Glucoside), sold by SEPPIC, Montanov 202 (INCI: Arachidyl Alcohol (and) Behenyl Alcohol (and) Arachidyl Glucoside), Citrol GMS 40 (INCI: Glyceryl stearate), sold by CRODA, or Imwitor 960K (INCI: Glyceryl stearate), sold by BIESTERFELD, Imwitor 372P (INCI: Glyceryl stearate citrate), sold by BIESTERFELD, Glucate SS (INCI: Methyl Glucose Sesquistearate), sold by LUBRIZOL ADVANCED MATERIALS, Inc, plural stearic (Polyglyceryl-6 Distearate), Natragem E145 (INCI: Polyglyceryl-4 Laurate/Succinate (and) Aqua), sold by CRODA, Span 60 (INCI: sorbitan stearate), sold by SIGMA ALDRICH.

Among the sorbitan esters, mention will for example be made of the product Span 20.

Among the fatty alcohols, mention will for example be made of the product sold under the name Promulgen D (INCI: Cetearyl alcohol (and) Ceteareth-20).

The W/O emulsifier may be advantageously selected from the emulsifying systems consisting of a cyclodextrin and a water-in-oil emulsifier of natural origin, such as the emulsifying system marketed by Roquette Frères under the name Beauté by Roquette® DS 146.

The W/O emulsifier may also be selected from the emulsifying systems consisting of modified starches and natural gums, such as the emulsifying system marketed by Roquette Frères under the name Beauté by Roquette® DS 421.

The water-in-oil (W/O) emulsifier is an emulsifying agent of HLB less than 8 and selected from non-ethoxylated polyol fatty esters, and especially from non-ethoxylated fatty esters of glycerol, of polyglycerols, of sorbitol, of sorbitan, of anhydrohexitols, such as in particular isosorbide, mannitol, xylitol, erythritol, maltitol, sucrose, glucose, polydextrose, hydrogenated glucose syrups, dextrins and hydrolyzed starches.

The emulsifying agent(s) are present in the composition according to the invention at a content ranging from 0.5% to 20%, preferably from 1 to 15%, preferably from 2 to 12%, by weight relative to the total weight of the composition.

### One or More Surfactants

According to one embodiment, the composition according to the invention comprises at least one surfactant selected from ionic surfactants, nonionic surfactants, cationic surfactants, anionic surfactants, or amphoteric or zwitterionic surfactants. These surfactants are selected for their detergent and foaming function.

According to another embodiment, the composition according to the invention comprises at least one surfactant selected from anionic surfactants, amphoteric surfactants or zwitterionic surfactants.

The anionic surfactants are chosen from carboxylic anionic surfactants, sulfate-based anionic surfactants, sulfonate surfactants, phosphate anionic surfactants, and mixtures thereof, preferably from sulfonate anionic surfactants, carboxylic anionic surfactants, and mixtures thereof.

“Anionic surfactant” is intended to mean a surfactant which only comprises anionic groups as ionic or ionizable groups. In the present description, an entity is described as being “anionic” when it has at least one permanent negative charge or when it can be ionized into a negatively charged entity, under the conditions of use of the composition of the invention (medium, pH for example) and not comprising any cationic charge. “Sulfate-based anionic surfactant” means an anionic surfactant comprising at least one sulfate function (—OSO₃H or —OSO₃⁻), and may optionally further comprise one or more other functions derived from acids, such as carboxylic acid or carboxylate functions (—COOH or —COO⁻), sulfonate functions (—SO₃H or —SO₃⁻) and/or phosphate functions. By way of example, alkyl sulfates, alkyl ether sulfates, alkylamidoether sulfates, alkylarylpolyethersulfates, monoglyceride sulfates, and also the salts of these compounds, are sulfate-based anionic surfactants. The alkyl groups of these compounds cited by way of example comprise 6 to 30 carbon atoms, and the aryl group denotes a phenyl or benzyl group. These compounds cited by way of example may be polyoxyalkylenated, in particular polyoxyethylenated, and comprise from 1 to 50 ethylene oxide units. “Non-sulfate-based anionic surfactant” is intended to mean a surfactant which does not fall within the definition of “sulfate-based anionic surfactant” as defined above.

It is also intended, according to the invention, that:
the carboxylic anionic surfactants comprise at least one carboxylic or carboxylate function (—COOH or —COO⁻), but do not comprise a sulfonic or sulfonate function (—SO₃H or —SO₃⁻), nor a sulfate function (—OSO₃H or —OSO₃⁻);
the sulfonate anionic surfactants comprise at least one sulfonic or sulfonate function (—SO₃H or —SO₃⁻), and may optionally further comprise one or more carboxylic or carboxylate (—COOH or —COO⁻) and/or phosphate functions, but do not comprise a sulfate function (—OSO₃H or —OSO₃⁻).
the phosphate anionic surfactants comprise at least one phosphoric or phosphate function (—OPO₃H₂ or —OPO3²⁻), but do not comprise a carboxylic or carboxylate function (—COOH or —COO⁻), nor a sulfonic or sulfonate function (—SO₃H or —SO₃⁻), nor a sulfate function (—OSO₃H or —OSO₃⁻).

In other words:
an anionic surfactant comprising at least one sulfate function (—OSO₃H or —OSO₃⁻), and at least one carboxylic or carboxylate function (—COOH or —COO⁻), is considered, within the meaning of the invention and unless otherwise indicated, as a sulfate-based anionic surfactant;
an anionic surfactant comprising at least one sulfate function (—OSO₃H or —OSO₃⁻), and at least one sulfonic or sulfonate function (—SO₃H or —SO₃⁻), is considered, within the meaning of the invention and unless otherwise indicated, as a sulfate-based anionic surfactant;
an anionic surfactant comprising at least one sulfonic or sulfonate function (—SO₃H or —SO₃⁻), and at least one carboxylic or carboxylate function (—COOH or —COO⁻), is considered, within the meaning of the invention and unless otherwise indicated, as a non-sulfated sulfonate anionic surfactant; • an anionic surfactant comprising at least one sulfate function (—OSO₃H or —OSO₃⁻), and at least one sulfonic or sulfonate function (—SO₃H or —SO₃⁻), and at least one carboxylic or carboxylate function (—COOH or —COO⁻), is considered, within the meaning of the invention and unless otherwise indicated, as a sulfate-based anionic surfactant.

The carboxylic anionic surfactants can be chosen from the following compounds: acylglycinates, acyllactylates, acylsarcosinates, acylglutamates, alkyl-D-galactoside-uronic acids, alkyl ether carboxylic acids, alkyl(aryl) ether carboxylic acids, alkyl(amido) ether carboxylic acids; and the salts of these compounds. The alkyl and/or acyl groups of these compounds comprise from 6 to 30 carbon atoms, more preferentially from 8 to 28, more preferentially still from 10 to 24, even better still from 12 to 22 carbon atoms. The aryl group preferably denotes a phenyl or benzyl group. These compounds may be polyoxyalkylenated, in particular polyoxyethylenated, and then preferably comprise from 1 to 50 ethylene oxide units, better still from 2 to 10 ethylene oxide units. It is also possible to use C6-C24 alkyl monoesters and polyglycoside-polycarboxylic acids such as C6-C24 alkyl polyglycoside citrates, C6-C24 alkyl polyglycoside tartrates, and salts thereof.

According to one embodiment, the carboxylic anionic surfactants are chosen from:
C6-C24, or even C12-C20 acylglutamates, such as stearoylglutamates, and in particular sodium stearoyl glutamate or disodium cocoylglutamates, in particular sodium or disodium cocoyl glutamate;
acylsarcosinates, in particular C6-C24 or even C12-C20 acylsarcosinates, such as cocoyl sarcosinates and in particular sodium cocoyl sarcosinate, lauroyl sarcosinates and in particular sodium lauroyl sarcosinate, palmitoylsarcosinates, and in particular sodium palmitoylsarcosinate;
acyllactylates, in particular C12-C28 or even C14-C24 acyllactylates, such as behenoyllactylates, and in particular sodium behenolyl lactylate, (iso) stearoyl lactylates, and in particular sodium (iso) stearoyl lactylate;
acylglycinates, in particular C6-C24 or C12-C20 acylglycinates, such as cocoyl glycinates and in particular sodium cocoyl glycinate;
alkyl(C6-C30) ether carboxylic acids, in particular alkyl(C6-C24) ether carboxylic acids;
alkyl(C6-C30) arylether carboxylic acids, in particular alkyl(C6-C24) arylether carboxylic acids;
alkyl(C6-C30)amidoether carboxylic acids, in particular alkyl(C6-C24)amidoether carboxylic acids;
and mixtures thereof; and in particular in the form of alkali or alkaline earth metal salts, ammonium, or aminoalcohol.

The anionic sulfonate surfactants can be selected from the following compounds: alkylsulfonates, alkylamidesulfonates, alkylarylsulfonates, C6-C24 alkyl polyglycoside sulfosuccinates, alpha-olefin sulfonates, kerosene sulfonates, alkylsulfosuccinates, alkyl ether sulfosuccinates, alkylamidesulfosuccinates, alkylsulfoacetates, N-acyltaurates, acyl isethionates; alkyl sulfolaurates; and salts of these compounds; the alkyl groups of these compounds containing from 6 to 30 carbon atoms, in particular from 12 to 28, even better from 14 to 24, or even from 16 to 22, carbon atoms; the aryl group preferably designating a phenyl or benzyl group; these compounds may be polyoxyalkylenated, in particular polyoxyethylenated and in such case preferably containing from 1 to 50 ethylene oxide units, better still from 2 to 10 ethylene oxide units.

According to one embodiment, the anionic sulfonate surfactants are chosen from: C6-C24, in particular C12-C20 alkyl sulfosuccinates, especially lauryl sulfosuccinates; C6-C24, in particular C12-C20 alkyl ether sulfosuccinates; (C6-C24) acyl isethionates, preferably (C12-C18) acyl isethionates; alpha-olefin sulfonates; and mixtures thereof; and in particular in the form of alkali or alkaline-earth metal, ammonium or aminoalcohol salts.

According to one embodiment, the anionic surfactants may be chosen from C6-C24 alkyl monoesters and polyglycoside-dicarboxylic acids such as alkyl glucoside citrates, alkyl polyglycoside tartrates and alkyl polyglycosidesulfosuccinates, alkylsulfosuccinamates, acyl isethionates and N-acyltaurates, the alkyl or acyl group of all these compounds preferably comprising from 12 to 20 carbon atoms. Another group of anionic surfactants that can be used in the compositions of the present invention is that of acyllactylates, the acyl group of which comprises from 8 to 20 carbon atoms. Additionally, other examples include alkyl-D-galactosideuronic acids and their salts, as well as polyoxyalkylenated (C6-C24 alkyl) ether carboxylic acids, polyoxyalkylenated (C6-24 alkyl) (C6-C24 aryl) ether carboxylic acids, polyoxyalkylenated (C6-24 alkyl)amidoether carboxylic acids and their salts, in particular those with 2 to 50 ethylene oxide units, and mixtures thereof.

The phosphate anionic surfactants are chosen from: C6-C24 alkyl phosphates, in particular C12-C20 alkyl phosphates; C6-C24 alkyl ether phosphates, in particular C12-C20 alkyl ether phosphates; and mixtures thereof.

The amphoteric or zwitterionic surfactants can be selected from the secondary, tertiary or quaternary aliphatic amine derivatives, wherein the aliphatic group is a linear or branched chain comprising from 8 to 22 carbon atoms and containing at least one anionic group such as, for example, a carboxylate, sulfonate, sulfate, phosphate or phosphonate group. Mention may also be made of: alkyl(C8-C20) betaines, for example cocobetaine, sulfobetaines, alkyl(C8-C20) sulfobetaines, alkyl(C8-C20)amidoalkyl(C3-C8) betaines, for example cocamidopropylbetaine; or alkyl(C8-C20)amidoalkyl(C6-C8) sulfobetaines.

Cationic surfactants can be selected from Cetrimonium Chloride sold under the Microcare Quat CTC 30 reference.

The surfactant(s) will be present in the composition according to the invention at a content ranging from 0.1% to 40%, preferably from 0.5 to 30%, preferably from 1 to 20%, preferably from 2 to 12% by weight relative to the total weight of the composition.

### One or More Film-Forming Agents

The film-forming agent may be selected from polymers of natural origin such as hydroxypropyl methyl cellulose (HPMC), hydroxypropyl cellulose (HPC), or synthetic polymers such as polyvinyl alcohol (PVA), and/or plasticizers other than polyols, such as polyethylene glycol, triethylcitrate, polysorbate, or waxes such as Carnauba wax, or hydrogenated castor oil.

Another film-forming agent of plant origin is Beauté by Roquette® ST 720 hydroxypropylated pea starch, sold by ROQUETTE.

The film-forming agent(s) are present in the composition according to the invention at a content ranging from 0.1% to 20%, preferably from 0.5 to 15%, preferably from 1 to 12%, by weight relative to the total weight of the composition.

### One or More Colorants

The composition according to the invention may further comprise at least one dyestuff selected from water-soluble or liposoluble dyes, fillers with the effect of coloring and/or opacifying the composition and/or coloring the skin or the hair or the oral mucosa, such as pigments, nacres, lakes (water-soluble dyes adsorbed on an inert mineral support) and mixtures thereof. These dyestuffs may be optionally surface-treated by a hydrophobic agent such as silanes, silicones, fatty acid soaps, C9-15 fluoroalcohol phosphates, acrylate/dimethicone copolymers, C9-15 fluoroalcohol phosphate/silicon mixed copolymers, lecithins, carnauba wax, polyethylene, chitosan and optionally amino acids which could be acylated such as lauroyl lysine, disodium stearoyl glutamate and aluminum acyl glutamate, phytic acid. Another natural colorant is caramel colorant Beauté by Roquette® CC 001, sold by ROQUETTE.

“Pigments” means white or colored, mineral or organic particles intended to color and/or opacify the cosmetic composition.

Among the pigments, mention will be made of mineral or organic, natural or synthetic pigments. Examples of pigments are especially iron, titanium or zinc oxides, and also composite pigments and goniochromatic, pearlescent, interference, photochromic or thermochromic pigments, with this list being non-limiting.

Among the pigments surface-treated with lecithins are UNIPURE WHITE LC981 HLC (INCI: CI 77891 (and) Hydrogenated Lecithin, UNIPURE YELLOW LC182 HLC (INCI: CI 77492 (and) Hydrogenated Lecithin), UNIPURE BLACK LC989 HLC (INCI: CI 77499 (and) Hydrogenated Lecithin, UNIPURE RED LC381 (INCI: CI 77491 (and) Hydrogenated Lecithin) from the company Sensient Cosmetic Technologies.

Among the pigments surface-treated with phytic acid are Unipure White LC 985 PHY (INCI: CI 77891 (and) Phytic Acid (and) Sodium Hydroxide), Unipure Yellow LC 188 PHY (INCI: CI 77492 (and) Phytic Acid (and) Sodium Hydroxide), Unipure Red LC 388 PHY (INCI: CI 77491 (and) Phytic Acid (and) Sodium Hydroxide), Unipure Black LC 998 PHY (INCI: CI 77499 (and) Phytic Acid (and) Sodium Hydroxide), from the company Sensient Cosmetic Technologies.

“Nacres” is intended to mean any iridescent or non-iridescent colored particles which have a color effect by optical interference.

Among nacres, mention will be made of titanium mica covered with metal oxide, such as iron oxide or titanium oxide.

According to one embodiment, the make-up and/or skincare composition according to the invention comprises at least colored particles, preferentially pigments such as iron oxide pigments and/or nacres.

The colorant(s) are present in the composition according to the invention at a content ranging from 0.05% to 20%, preferably from 0.1% to 15%, preferably from 0.5 to 12%, by weight relative to the total weight of the composition.

### One or More Sensory Filler(s) or Powder(s)

This term is intended to mean particles of any form (especially spherical or lamellar), mineral or organic, which are insoluble in the composition. Examples of sensory fillers or powders are talc, boron nitride, starch such as the Beauté by Roquette® ST 005 and Beauté by Roquette® ST 012 granular starches sold by ROQUETTE, polyamides, silicone resins, silicone elastomer powders and acrylic polymer powders, in particular powders of poly(methyl methacrylate) or powders of styrene acrylate copolymer (Sunsphere Powders from Dow).

The filler(s) are present in the composition according to the invention at a content ranging from 0.5% to 20%, preferably from 1 to 15%, preferably from 3 to 12%, by weight relative to the total weight of the composition.

### One or More Sensory Agent(s)

Sensory agents can be used to modify the sensory profile of a cosmetic or dermatological composition, making it more pleasant to use on the skin. Sensory agents can be chosen from modified starches such as carboxymethylated starches, e.g. Roquette's Beauté by Roquette ST 118, silicas or talcs, or mixtures thereof. These sensory agents provide a soft touch for solid compositions, or a cushioning effect for compositions in liquid or gel form.

The sensory agent(s) are present in the composition according to the invention at a content ranging from 0.1% to 20%, preferably from 1 to 15%, preferably from 3 to 12%, by weight relative to the total weight of the composition.

### One or More Active Ingredients

In addition to hyperbranched dextrins, the cosmetic composition may comprise one or more active ingredients.

These active ingredients can be of a wide range of chemical types, since, as the applicant has noted, hyperbranched dextrin, preferentially hyperbranched and hydrogenated dextrin, is fairly inert, with low reactivity and excellent compatibility with most active ingredients used in cosmetic or even dermatological compositions. These active ingredients may be chosen from moisturizing, emollient, film-forming, barrier, anti-pollution, tensing, anti-aging, anti-oxidant, exfoliating, collagen stimulating, anti-acne, sebum reducing, blood circulation stimulating, refreshing, antibacterial, antifungal, anti-inflammatory, soothing, anti-irritant, healing, slimming, pigmenting, coloring, mattifying and perfuming agents.

Among soothing agents, those extracted from plants are preferentially used. By way of illustration, C-glycosides such as those disclosed in FR2902998 are used as soothing agents. Natural products such as ceramides, sweet almond or chamomile oils, extracts of mimosa tenuiflora (tepezcohuite), aloe vera, licorice (glycyrrhetinic acid), calendula, St. John's wort, oats, linden, magnolia, marshmallow or mallow are also particularly effective soothing agents.

Preferentially, hyperbranched, preferentially hyperbranched and hydrogenated dextrins can be combined with an anti-inflammatory cosmetic active ingredient or another soothing active ingredient.

The active ingredient(s) are present in the composition according to the invention at a content ranging from 0.05% to 20%, preferably from 0.1 to 10%, preferably from 0.5 to 6%, by weight relative to the total weight of the composition.

### EXAMPLES

### Example 1: Clinical Study of Discomfort Induced by Sodium Lauryl Sulfate

The aim of the study was to determine the effect of a hyperbranched and hydrogenated dextrin, sold under the reference Nutriose® HM 06 by Roquette, on sensations of discomfort created by exposure of the skin of the inner forearm of volunteers to a skin-aggressive product, sodium lauryl sulfate (SLS), applied by a patch at a concentration in water of 10%.

### Volunteers and Products

This non-invasive, single-center study was carried out on forty male volunteers aged between 30 and 55 years of age with sensitive skin (based on self-reporting). The inclusion criteria for volunteers are shown in the table.

TABLE 1

| Criteria | |
|---|---|
| Gender | Male |
| Phototype | I to III |
| Age | From 30 to 55 (divided into 3 groups |
| | of homogeneous average groups) |
| Sensitive skin | Yes, based on volunteers' reporting |

Volunteers were divided into two homogeneous groups of 20 volunteers, and each group received a lotion according to the table below.

### Lotion Compositions

TABLE 2

| | | Volunteer group No. | |
|---|---|---|---|
| | | 1 | 2 |
| Ingredient/Supplier | INCI | Placebo | Lotion |
| | | lotion | according to |
| | | | the invention |
| Aqua | | 96.8 | 91.8 |
| Nutriose ® HM 06 | Not available | 0 | 5 |
| Cosmedia ® SP | Sodium | 0.2 | 0.2 |
| | polyacrylate | | |
| Caprylis/Labrafac | Caprylic capric | 2 | 2 |
| cc | triglycerides | | |
| Microcare ® PHC | Phenoxyethanol | 1.00 | 1.00 |
| | and glycerin | | |
| Citric acid | | Q.s. pH | Q.s. pH |
| | | 6 +/− 0.3 | 6 +/− 0.3 |

### Study Protocol

At the start of the study (day 0), SLS 10% patches were placed on the two delineated areas on the forearms of each volunteer. Volunteers kept the patches on for 20 hours, and removed them 4 hours before the Day 1 self-assessment, so that on Day 1, self-assessments were done on both forearms of all volunteers. Immediately after the Day 1 measurement, each group applied its lotion to the area exposed to the SLS patch on the right forearm, which is the treated area, and then performed a self-assessment 5, 10 and 30 minutes after this application. The left forearm was not treated with any lotion; this is the untreated area.

From day 1 to day 6, volunteers applied the same lotion to the SLS-exposed area of the right forearm every evening. The left forearm received no treatment. On days 2, 3, 4 and 7, self-assessments of the area treated with the lotion and untreated area were carried out.

### Self-Assessment

Volunteers rated their own sensations of discomfort, warmth and tightness for both the treated and untreated areas, on semi-structured scales ranging from 1 to 10, with 1 corresponding to “not intense” and 10 to “very intense”.

### Results

TABLE 3

| | Placebo group | | Invention Group | |
|---|---|---|---|---|
| Warm | Treated | Untreated | Treated | Untreated |
| sensation | area | area | area | area |
| Day 1 | 2.40 | 2.30 | 1.75 | 1.65 |
| Day 1 + 5 min | 1.95 | 1.95 | 1.80 | 1.60 |
| Day 1 + 10 min | 1.90 | 1.80 | 1.45 | 1.55 |
| Day 1 + 30 min | 1.60 | 1.65 | 1.50 | 1.55 |
| Day 2 | 1.35 | 1.50 | 1.60 | 1.85 |
| Day 3 | 1.25 | 1.20 | 1.50 | 1.70 |
| Day 4 | 1.30 | 1.20 | 1.30 | 1.50 |
| Day 7 | 1.15 | 1.15 | 1.10 | 1.45 |

The table describes the average values of the self-assessment of the feeling of warming.

TABLE 4

| Sensation of | Placebo group | | Invention Group | |
|---|---|---|---|---|
| tightness | Treated | Untreated | Treated | Untreated |
| Day 1 | 2.10 | 2.20 | 1.85 | 1.60 |
| Day 1 + 5 min | 1.80 | 2.00 | 1.45 | 1.55 |
| Day 1 + 10 min | 1.85 | 1.75 | 1.50 | 1.45 |
| Day 1 + 30 min | 1.60 | 1.55 | 1.35 | 1.45 |
| Day 2 | 1.35 | 1.40 | 1.30 | 1.45 |
| Day 3 | 1.25 | 1.25 | 1.25 | 1.30 |
| Day 4 | 1.35 | 1.15 | 1.10 | 1.20 |
| Day 7 | 1.15 | 1.15 | 1.10 | 1.10 |

The table shows the average values for the self-assessment of the feeling of tightness.

To compare changes in sensations of warmth and tightness between the different groups, the variations in these sensations were calculated between the different measurement times, for example for a treated area of skin:

$$[ Math . 1 ]$$

$$Δ Treated ( ( Day x ) - Day 1 ) = ( sensation assessed in treated area at day \\ x ) - sensation assessed in treated area at day 1 ) , for both groups .$$

TABLE 5

| | Warmth | | Tightness | |
|---|---|---|---|---|
| | Placebo | Invention | Placebo | Invention |
| | group | group | group | group |
| ΔTreated((Day 1 + 5 min) − Day 1) − | −0.10 | 0.10 | −0.10 | −0.35 |
| ΔUntreated ((Day 1 + 5 min) − Day 1) | | | | |
| ΔTreated((Day 1 + 10 min) − Day 1) − | 0.00 | −0.20 | 0.20 | −0.20 |
| ΔUntreated ((Day 1 + 10 min) − Day 1) | | | | |
| ΔTreated((Day 1 + 30 min) − Day 1) − | −0.15 | −0.15 | 0.15 | −0.35 |
| ΔUntreated ((Day 1 + 30 min) − Day 1) | | | | |
| ΔTreated((Day 2) − Day 1) − | −0.25 | −0.35 | 0.05 | −0.40 |
| ΔUntreated ((Day 2) − Day 1) | | | | |
| ΔTreated((Day 3) − Day 1) − | −0.05 | −0.30 | 0.10 | −0.30 |
| ΔUntreated ((Day 3) − Day 1) | | | | |
| ΔTreated((Day 4) − Day 1) − | 0.00 | −0.30 | 0.30 | −0.35 |
| ΔUntreated ((Day 4) − Day 1) | | | | |
| ΔTreated((Day 7) − Day 1) − | −0.10 | −0.45 | 0.10 | −0.25 |
| ΔUntreated ((Day 7) − Day 1) | | | | |

The table describes the average variations in sensations of heating and tightness over time between treated and untreated areas of skin.

Nutriose® HM 06 hyperbranched dextrin reduces SLS-induced warming sensations as early as 10 minutes compared with the placebo group, and this reduction is increased and maintained for up to 7 days.

Nutriose® HM 06 hyperbranched dextrin reduces SLS-induced tightness as early as 5 minutes compared with the placebo group, and this reduction is maintained on average for up to 4 days.

The reduction in sensations of skin warming and tightness by application of Nutriose® HM 06 hyperbranched dextrin showed that this dextrin was able to soothe cutaneous reactions caused by the SLS 10% patch. Nutriose® HM 06 hyperbranched and hydrogenated dextrin is therefore soothing.

### Example 2: Cosmetic Product Formulations Comprising a Hyperbranched and Hydrogenated Dextrin According to the Object of the Application

Roquette's Nutriose® HM 06 hyperbranched and hydrogenated dextrin, due to its particular chemical nature disclosed herein before, has, as the applicant has observed, very little or no reactivity with most of the active ingredients used in cosmetic and dermatological compositions, and is thereby highly compatible and easy to use for the formulator.

In fact, it can act as a soothing agent in a large number of formulations, such as those which follow and which are given by way of illustration and not by way of limitation.

### After-Sun Cream

TABLE 6

| | | | | % |
|---|---|---|---|---|
| Phase | INCI | Trade name | Supplier | w/w |
| A | Aqua | Demineralized water | / | To 100 |
| | Sorbitol | Beauté by | Roquette | 5 |
| | | Roquette ® PO 071 | | |
| | Not available | Nutriose ® HM 06 | Roquette | 5 |
| | Xanthan gum | Xanthan gum | JBL | 0.3 |
| | | FNCSP- PC | | |
| | Polyglyceryl-10 Laurate | Emulpharma Eco 10 | Res Pharma | 3 |
| B | Hydrogenated Argania | Nat Organic Argan | Naturochim | 2 |
| | Spinosa Kernel Oil | Wax | | |
| | Butyrospermum Parkii | Shea butter | Aromazone | 3 |
| | (Shea) Butter | | | |
| | Jojoba Esters and | Acticire MB | Gattefossé | 5 |
| | Helianthus Annuus | | | |
| | (Sunflower) Seed Wax | | | |
| | (and) Acacia Decurrens | | | |
| | Flower Wax (and) | | | |
| | Polyglycerin-3 | | | |
| | Argania Spinosa (argan) | Deodorized virgin | Cooper | 12 |
| | Kernel Oil | argan oil | | |
| | Tocopherols (mixed), | Vitamin E | Aromazone | 0.2 |
| | helianthus annuus seed | | | |
| | oil | | | |
| C | Aluminium starch | Beauté by | Roquette | 3 |
| | octenylsuccinate | Roquette ® ST 012 | | |
| D | Aqua | Demineralized water | / | 5 |
| | Potassium sorbate | Potassium sorbate | Cooper | 0.4 |
| | Sodium benzoate | Sodium benzoate | Cooper | 0.4 |

Preparation protocol: phase A and phase B were prepared at 70° C. in two separate beakers. Phase B was then emulsified in phase A under high-shear stirring for 10 minutes. This was followed by cooling to 20° C.+/−2° C. Phase C was then added with stirring, followed by phase D. Finally, the pH was adjusted to 6. A white cream was obtained, with a Brookfield viscosity of 3500 mPa·s+/−700 (spindle No. 4 at 20 rpm).

### After-Shave Gel

TABLE 7

| | | | | % |
|---|---|---|---|---|
| Phase | INCI | Trade name | Supplier | w/w |
| A | Aqua | Demineralized water | / | To 100 |
| | Isosorbide | Beauté by Roquette ® | Roquette | 5 |
| | | PO 500 | | |
| | Starch acetate, | Beauté by Roquette ® | Roquette | 3 |
| | hydroxyethylcellulose | DS 112 | | |
| | and xanthan gum | | | |
| | Not available | Nutriose ® HM 06 | Roquette | 5 |
| B1 | Aqua | Demineralized water | / | 5 |
| | Potassium sorbate | Potassium sorbate | Cooper | 0.4 |
| B2 | Gluconic acid, | Beauté by Roquette ® | Roquette | 1 |
| | caprylyl/capryl | LS 007 | | |
| | glucoside, cymbobogon | | | |
| | citratus leaf oil | | | |
| C | Sodium hydroxide18 | Sodium hydroxide18 | / | qs pH |
| | | | | 5.5-6 |

### Preparation Protocol: Isosorbide, Nutriose

HMC 06 were mixed beforehand in the quantity of water required for phase A, then Beauté by Roquette DS 112 starch was dispersed at 80° C. for 30 to 40 minutes. Phases B1 and B2 were then added successively, and the pH adjusted to 6. A translucent gel was obtained, with a Brookfield viscosity of 3500 mPa·s+/−700 (spindle No. 4 at 20 rpm).

### Soothing Oil-In-Water Emulsion

TABLE 8

| | | | | % |
|---|---|---|---|---|
| Phase | INCI | Trade name | Supplier | w/w |
| A1 | Aqua | Demineralized water | / | 61.93 |
| | Not available | Nutriose ® HM 06 | Roquette | 5.00 |
| A2 | Xanthan gum | Keltrol CG | CP Kelco | 0.30 |
| | Microcrystalline | Tabulose | Roquette | 1.00 |
| | cellulose, cellulose gum | | | |
| A3 | Cyclodextrin, Sorbitol, | Beauté by | Roquette | 5.00 |
| | Polyglyceryl-3 | Roquette ® DS 146 | | |
| | diisostearate | | | |
| B | Diheptyl Succinate, | Lexfeel N350 | Inolex | 20.00 |
| | Capryloyl | | | |
| | Glycerin/Sebacic Acid | | | |
| | Copolymer | | | |
| C | Zea Mays Starch | Beauté by | Roquette | 5.00 |
| | | Roquette ® ST 005 | | |
| D | Gluconic acid, | Beauté by | Roquette | 1.00 |
| | caprylyl/Capryl | Roquette ® LS 007 | | |
| | glucoside, cymbopogon | | | |
| | citratus leaf oil | | | |
| E | Potassium sorbate | Microcare KS | Thor | 0.40 |
| F | Fragrance | Delicious apple IPO | LR | 0.30 |
| | Color E 133 | FDC blue 1 (2% sol. | | 0.07 |
| | | aq.) | | |

Preparation protocol: Nutriose HMC 06 was added to half the volume of water required for formulation at 45° C., stirred with a deflocculator at 500 rpm until a clear solution was obtained. In addition, the ingredients of phase A2 were mixed in half the volume of water required for formulation, with stirring at 500-1000 rpm. Phase A1 was then mixed with phase A2, and phase A3 added with stirring at 1500 rpm for 10 minutes. Phase B was also prepared by heating to 45° C. Phase B was then emulsified in phase A1+A2+A3 with vigorous stirring at 2000-3000 rpm for 15 minutes at 45° C. This was cooled to 20° C.+/−2° C., phases C, D, E and F were successively added with 500 rpm stirring, and pH adjusted to 5.3+/−0.2. The result is a blue emulsion with a Brookfield viscosity of 4500-5500 mPa·s (spindle No. 3 at 20 rpm).

### Soothing and Repairing Hand Cream

TABLE 9

| | | | | % |
|---|---|---|---|---|
| Phase | INCI | Trade name | Supplier | w/w |
| A | Aqua | Water | — | 60.3 |
| | Not available | Nutriose ® HM 06 | Roquette | 5 |
| B | Starch Acetate, Starch | Beauté by | Roquette | 3 |
| | sodium octenylsuccinate, | Roquette | | |
| | Guar gum, Tara gum, | DS 421 | | |
| | Xanthan gum | | | |
| C | Prunus amygdalus dulcis | Sweet almond oil | Cooper | 30 |
| | oil | | | |
| | Fragrance | Nola Fresh Juice | L.R. Flavours | 0.2 |
| | | | & Fragrances | |
| | Benzyl alcohol | Microcare alcohol | Thor | 0.5 |
| | | BNA | | |
| D | Gluconic acid, | Beauté by | Roquette | 1 |
| | caprylyl/Capryl glucoside, | Roquette LS 007 | | |
| | cymbopogon citratus leaf | | | |
| | oil | | | |

Preparation protocol: Nutriose HMC 06 was added to the volume of water required for formulation at 45° C., stirred with a deflocculator at 500 rpm until a clear solution was obtained, then cooled to 20° C.+/−2° C. Beauté by Roquette DS 421 was then dispersed in phase A under stirring with a deflocculator at 1000 rpm for about 10 minutes to hydrate the ingredient, which was visually observed by its opalescence. Phase C was prepared separately. At ambient temperature (20° C.+/−2° C.), phase C was added to phase A+B slowly and with stirring using a deflocculator at 2000-3000 rpm, then stirring was maintained for 10 minutes. Finally, phase D was added with stirring at 1000-2000 rpm, then adjusted to pH 4.5-4.7. A yellow cream was obtained, with a Brookfield viscosity of 8800 mPa·s (spindle 3 at 20 rpm).

### Soothing Cleansing Cream (TO-027-001)

TABLE 10

| | | | | % |
|---|---|---|---|---|
| Phase | INCI | Trade name | Supplier | w/w |
| A1 | Aqua | Demineralized | — | 28.3 |
| | | Water | | |
| | Not available | Nutriose ® HM 06 | Roquette | 5 |
| | Xanthan Gum | Keltrol | CP Kelco | 0.7 |
| A2 | Cyclodextrin, Sorbitol, | Beauté by | Roquette | 6 |
| | Polyglyceryl-3 diisostearate | Roquette DS 146 | | |
| B | Helianthus annuus seed oil | Sunflower oil | Cooper | 35 |
| C | Glycerin (and) | Microcare PHC | Thor | 1 |
| | Phenoxyethanol (and) | | | |
| | Chlorphenesin | | | |
| D | Sodium Lauryl ether Sulfate | Texapon NSO UP | BASF | 7 |
| | Coco Glucoside | Pureact Gluco C | Innospec | 5 |
| | Disodium Laureth | Rewopol SB FA | Evonik | 5 |
| | Sulfosuccinate | 30 B | | |
| | Cocamidopropyl Betaine | Dehyton PK45 | BASF | 7 |

### Soothing Hair Conditioning Gel (HC-040-001)

TABLE 11

| | | | | % |
|---|---|---|---|---|
| Phase | INCI | Trade name | Supplier | w/w |
| A1 | Aqua | Demineralized | / | 81 |
| | | water | | |
| | Maltitol | Beauté by | Roquette | 5 |
| | | Roquette ® PO 455 | | |
| | Not available | Nutriose ® HM 06 | Roquette | 5 |
| A2 | Starch acetate, | Beauté by | Roquette | 5 |
| | Hydroxyethyl- cellulose, | Roquette ® DS 112 | | |
| | Xanthan gum | | | |
| A3 | Cetrimonium Chloride | Microcare Quat | Thor | 3 |
| | | CTC 30 | | |
| B | Phenoxyethanol, | Microcare PHC | Thor | 1 |
| | Chlorphenesin, Glycerin | | | |

### Soothing Hair Conditioning Cream (HC-040-002)

TABLE 12

| | | | | |
|---|---|---|---|---|
| | | | | % |
| Phase | INCI | Trade name | Supplier | w/w |

| | | | |
|---|---|---|---|
| A1 | Aqua | Demineralized water | / |
| | Maltitol | Beauté by | Roquette |
| | | Roquette ® PO 455 | |
| | Not available | Nutriose ® HM 06 | Roquette |
| A2 | Starch acetate, | Beauté by | Roquette |
| | Hydroxyethyl- cellulose, | Roquette ® DS 112 | |
| | Xanthan gum | | |
| A3 | Cetrimonium Chloride | Microcare Quat CTC | Thor |
| | | 30 | |
| B | Caprylic capric triglycerides | Caprylis | Aroma |
| | | | Zone |
| C | Phenoxyethanol, | Microcare PHC | Thor |
| | Chlorphenesin, Glycerin | | |

### Soothing Elixir (SC-073-003)

TABLE 13

| | | | | % |
|---|---|---|---|---|
| Phase | INCI | Trade name | Supplier | w/w |
| A | | Aqua | | 84.20 |
| | Sorbitol | Beauté by | Roquette | 3.00 |
| | | Roquette ® PO 070 | | |
| | Not available | Nutriose ® HM 06 | Roquette | 5.00 |
| | Starch acetate, | Beauté by | Roquette | 4.00 |
| | Hydroxyethyl- cellulose, | Roquette ® DS 112 | | |
| | Xanthan gum | | | |
| B | | tsubaki oil | | 1.00 |
| | | microcare PHC | | 0.30 |
| C | Gluconic acid, | Beauté by | Roquette | 1.5 |
| | caprylyl/Capryl glucoside, | Roquette ® LS 007 | | |
| | cymbopogon citratus leaf oil | | | |
| | Carboxymethyl starch | Beauté by | Roquette | 1 |
| | | Roquette ® ST 118 | | |
| | | NaOH QS pH 5.5 | | |

### Sulfated Surfactant-Free Shampoo (HC-031-002)

TABLE 14

| | | | | % |
|---|---|---|---|---|
| Phase | INCI | Trade name | Supplier | w/w |
| A1 | Water | Aqua | | 61.55 |
| | Not available | Nutriose ® HM 06 | Roquette | 5 |
| | Keltrol | Xanthan | | 1 |
| | Plantacare 2000 | Decyl glucoside | | 18 |
| A2 | | Sodium cocoyl | | 7 |
| | | glutamate | | |
| | plantacare 818 or puract gluco C | COCO- | | 2.25 |
| | | GLUCOSIDE | | |
| B | BBR PO 070 | Sorbitol | | 3 |
| | Gluconic acid, caprylyl/Capryl | Beauté by | Roquette | 1.5 |
| | glucoside, cymbopogon citratus | Roquette ® LS 007 | | |
| | leaf oil | | | |
| | | Beauté by | Roquette | 0.2 |
| | | Roquette ® CC 001 | | |
| C | Sodium chloride | NaCl | | 0.5 |

### Soothing Shampoo (HC-025-008)

TABLE 15

| | | | | % |
|---|---|---|---|---|
| Phase | INCI | Trade name | Supplier | w/w |
| A | Water | Aqua | — | 62.8 |
| | Not available | Nutriose ® HM 06 | Roquette | 5 |
| | Keltrol CG-SFT | xanthan gum | CP | 1 |
| | | | Kelco | |
| B | Plantacare 818 UP | Coco glucoside, aqua | BASF | 28.8 |
| | Imwitor 948 | Glyceryl oleate | BASF | 1 |
| D | Water | Aqua | — | 0.00 |
| | Gluconic acid, | Beauté by Roquette ® LS | Roquette | 1 |
| | caprylyl/Capryl glucoside, | 007 | | |
| | cymbopogon citratus leaf oil | | | |
| | Potassium Sorbate | Potassium Sorbate | Thor | 0.4 |

### After-Sun Cream (SC-073-011)

TABLE 16

| | | | | % |
|---|---|---|---|---|
| Phase | INCI | Trade name | Supplier | w/w |
| | Aqua | Aqua | | 83.83 |
| A | Sorbitol | Beauté by Roquette ® | Roquette | 3 |
| | | PO 070 | | |
| | Dextrin | Nutriose ® HM 06 | Roquette | 5 |
| | Starch acetate, | Beauté by Roquette ® | Roquette | 4 |
| | Hydroxyethyl cellulose, | DS 112 | | |
| | Xanthan gum | | | |
| B | Camellia Japonica | tsubaki oil | Jan dekker | 1 |
| | Seed Oil | | | |
| | Fragrance | Fico blanco e aloe | LR fragrance | 0.1 |
| | | | & flavours | |
| | Phenoxyethanol, | microcare PM2 | Thor | 0.5 |
| | Ethylparaben, | | | |
| | Methylparaben | | | |
| C | Gluconic acid, | Beauté by Roquette ® | Roquette | 1.5 |
| | Caprylyl/capryl | LS 007 | | |
| | glucoside, Cymbopogon | | | |
| | citratusleaf oil | | | |
| D | Sodium Carboxymethyl | Beauté by Roquette ® | Roquette | 1 |
| | starch | ST 118 | | |
| E | Aqua and CI 42090 | Unicert Blue 05601-J | Sensient | 0.07 |
| | | aqueous solution 2% | Cosmetic | |
| | | | Technologies | |
| F | Sodium Hydroxide | — | Cooper | Qs pH 5 |

Preparation protocol: Nutriose HM® 06 is dispersed in a mixture of water and Beauté by Roquette® PO 070 sorbitol, with gentle stirring. Beauté by Roquette® DS 112 is then added, and the product is heated to 80° C. with medium-speed stirring using a deflocculating paddle, and held at this temperature with stirring for 40 minutes to obtain a homogeneous gel. Cool to 25-30° C. with gentle stirring. Phase B is then added with medium stirring, and stirring is maintained for 5 minutes. Phase C is then added with medium stirring, followed by phase D with medium stirring, and stirred for 5 minutes. Phase E is added with gentle stirring. Finally, phase F is used to adjust the pH to 5.

### Solar Barrier Stick (SU-024-018))

TABLE 17

| | | | | % |
|---|---|---|---|---|
| Phase | INCI | Trade name | Supplier | w/w |
| A | Homosalate | Eusolex HMS | Merck | 5 |
| | Octocrylene | Eusolex OCR | Merck | 6 |
| | Butylmethoxydibenzoylmethane | Eusolex 9020 | Merck | 3 |
| | Bis-Ethylhexyloxyphenol | Tinosorb S | BASF | 5 |
| | Methoxyphenyl Triazine | | | |
| | Ethylhexyl Salicylate | Eusolex OS | Merck | 4 |
| | Hydrogenated Castor Oil | cutina HR | BASF | 2.5 |
| | Copernicia Cerifera Cera | Carnauba wax T1 | Koster | 3.5 |
| | | | Keunen | |
| | Glyceryl Behenate | Compritol 888 | Gatefosse | 5 |
| | Olus Oil, Hydrogenated | Cegesoft VP | BASF | 3.5 |
| | Vegetable Oil, Euphorbia | | | |
| | Cerifera (Candelilla) Wax | | | |
| | Cera alba | Beeswax white | Koster | 4 |
| | | | Keunen | |
| | Dicaprylyl Carbonate | Cetiol CC | BASF | 7 |
| | Behenyl Alcohol | Lanette 22 | BASF | 4 |
| | Theobroma Cacao Seed | Cocoa Butter RBD | Jan Dekker | 11 |
| | Butter | | | |
| | Zea Mays Starch | Beauté by | Roquette | 5 |
| | | Roquette ® ST 005 | | |
| | Tocopherol, Sunflower Oil | Bioxan SF T50 | BTSA | 1.6 |
| | Sodium gluconate | Beauté by | Roquette | 0.2 |
| | | Roquette ® GA 080 | | |
| | Diheptyl Succinate, Capryloyl | Lexfeel 20 NB | Inolex | 7.4 |
| | Glycerin/Sebacic Acid | | | |
| | Copolymer | | | |
| B | Aqua | Demineralized | / | 10 |
| | | water | | |
| | Dextrin | Nutriose ® HM 06 | Roquette | 2 |
| | Caramel | Beauté by | Roquette | 10.2 |
| | | Roquette ® CC | | |
| | | 001 | | |
| | Methylene Bis-Benzotriazolyl | Tinosorb M | BASF | 5 |
| | Tetramethylbutylphenol, | | | |
| | Aqua, Decyl Glucoside, | | | |
| | Propylene Glycol, Xanthan | | | |
| | Gum | | | |
| | Phenoxyethanol, | Microcare PHC | Thor | 1 |
| | Chlorphenesin, Glycerin | | | |
| | Fragrance, Coumarin | Vanilla Sky | L.R. Flavours | 0.1 |
| | | | & Fragrances | |
| | | | Industries | |
| | Cyclodextrin, Sorbitol, | Beauté by | Roquette | 4 |
| | Polyglyceryl-3 Diisostearate | Roquette ® DS 146 | | |

Preparation protocol: phase A ingredients are mixed under stirring and heated to 80° C. Phase B is then prepared with vigorous stirring using a deflocculating paddle (2000 rpm) until all ingredients are well dispersed. Phase A is cooled to 60° C., then added to phase B with gentle stirring. Holding at 60° C., the mixture is poured into a mold, then left to cool to 20° C. The result is a solid composition in stick form.

1. A non-therapeutic use of hyperbranched dextrins, preferentially hydrogenated hyperbranched dextrins, as a soothing agent, wherein said dextrins comprise at least 5% of 1-6 glucosidic bonds relative to the sum of 1-2, 1-3, 1-4 and 1-6 bonds.

2. The non-therapeutic use according to claim 1, wherein said dextrins have:
from 5% to 40% of 1-6 glucosidic bonds in relation to the sum of 1-2, 1-3, 1-4 and 1-6 bonds,
from 42 to 50% of 1-4 glucosidic bonds relative to the sum of 1-2, 1-3, 1-4 and 1-6 bonds,
from 1 to 20% of 1-3 glycosidic bonds relative to the sum of 1-2, 1-3, 1-4 and 1-6 bonds,
from 1 to 20% of 1-2 glycosidic bonds relative to the sum of 1-2, 1-3, 1-4 and 1-6 bonds.

3. The non-therapeutic use according to claim 1, wherein said dextrins have:
at least 5% of 1-6 glucosidic bonds relative to the sum of 1-2, 1-3, 1-4 and 1-6 bonds, preferentially at least 10%, more preferentially at least 12%, and most preferentially at least 15%
no more than 70% of 1-4 glycosidic bonds, relative to the sum of 1-2, 1-3, 1-4 and 1-6 bonds, preferentially no more than 60%, and most preferentially no more than 50%,
at least 1% of 1-3 glucosidic bonds relative to the sum of 1-2, 1-3, 1-4 and 1-6 bonds, preferentially at least 5%, more preferentially at least 10%, and most preferentially at least 20%,
at least 1% of 1-2 glucosidic bonds relative to the sum of 1-2, 1-3, 1-4 and 1-6 bonds, preferentially at least 5%, more preferentially at least 10%, and most preferentially at least 20%.

4. The non-therapeutic use according to claim 1, wherein the soothing agent is an agent for soothing cutaneous reactions and/or oral mucosal reactions.

5-7. (canceled)

8. The non-therapeutic use according to claim 1, wherein the cutaneous reactions are cutaneous reactions of the skin and/or scalp and are selected from warming, tightness, tingling, numbness, itching.

9. The non-therapeutic use according to claim 1, therein the oral mucosal reactions are selected from burning, warming, tightness, tingling, numbness and itching.

10. The non-therapeutic use according to claim 1, wherein the skin and/or scalp and/or oral mucosa are sensitive.

11. The non-therapeutic use according to claim 1, wherein the cutaneous reactions and/or oral mucosal reactions are induced by endogenous and/or exogenous stress.

12. The non-therapeutic use according to claim 1, wherein the exogenous stress is chosen from chemicals or formulation ingredients, mechanical aggression, environmental factors, thermal aggression, foods and beverages.

13. The non-therapeutic use according to claim 1, wherein the endogenous stress is chosen from hormonal secretions, age, stress and perspiration.

14. A method of soothing non-therapeutic care of the skin and/or scalp and/or oral mucosa comprising the application of hyperbranched dextrins, preferentially hydrogenated hyperbranched dextrins, to the skin and/or scalp and/or oral mucosa, wherein said dextrins comprise at least 5% of 1-6 glucosidic bonds relative to the sum of 1-2, 1-3, 1-4 and 1-6 bonds.

15. The non-therapeutic care method according to claim 14, wherein said dextrins have:
from 5% to 40% of 1-6 glucosidic bonds in relation to the sum of 1-2, 1-3, 1-4 and 1-6 bonds,
from 42 to 50% of 1-4 glucosidic bonds relative to the sum of 1-2, 1-3, 1-4 and 1-6 bonds,
from 1 to 20% of 1-3 glycosidic bonds relative to the sum of 1-2, 1-3, 1-4 and 1-6 bonds,
from 1 to 20% of 1-2 glycosidic bonds relative to the sum of 1-2, 1-3, 1-4 and 1-6 bonds.

16. The non-therapeutic care method according to claim 14 comprising the application of hyperbranched dextrins, preferentially hydrogenated hyperbranched dextrins, to the skin and/or scalp and/or oral mucosa exhibiting one or more cutaneous reactions.

17. The non-therapeutic care method according to claim 14, wherein hyperbranched dextrins, preferentially hydrogenated hyperbranched dextrins, are applied to the skin and/or scalp and/or oral mucosa in preparation for the application of a cosmetic or dermatological treatment or endogenous and/or exogenous stress.

18. A cosmetic composition comprising, in a cosmetically acceptable medium, hyperbranched dextrins, preferentially hydrogenated hyperbranched dextrins, wherein said dextrins comprise at least 5% of 1-6 glucosidic bonds relative to the sum of 1-2, 1-3, 1-4 and 1-6 bonds.

19-20. (canceled)
